(19) United States
(12) Patent Application Publication
(10) Pub. No.: US 2018/0185421 A1
(43) Pub. Date: Jul. 5, 2018

(54) COMPOSITION COMPRISING FECAL MICROBIOTA

(71) Applicants: TOKYO METROPOLITAN GOVERNMENT, Tokyo (JP); The University of Tokyo, Tokyo (JP); KEIO UNIVERSITY, Tokyo (JP)

(72) Inventors: Kazuhiko KAKIHANA, Tokyo (JP); Masahira Hattori, Tokyo (JP); Kenya Honda, Tokyo (JP); Hiroyoshi Nishikawa, Chiba (JP); Hidetoshi Morita, Okayama (JP); Kozue Takeshita, Tokyo (JP)

(21) Appl. No.: 15/688,465

(22) Filed: Aug. 28, 2017

(30) Foreign Application Priority Data
Aug. 29, 2016 (JP) 2016-166656

(51) Int. Cl. A61K 35/742; A61K 35/38; A61K 9/48; A61P 37/00

(52) U.S. Cl. CPC A61K 35/742; A61P 37/00; A61K 9/48; A61K 35/38

(57) ABSTRACT

The present invention provides a composition for preventing or treating a graft-versus-host disease, the composition comprising a fecal microbiota.

### FIELD OF THE INVENTION

The present invention relates to a composition comprising a fecal microbiota, specifically, a composition for preventing or treating gastrointestinal acute graft-versus-host disease.

### BACKGROUND ART

While allogeneic hematopoietic stem cell transplantation (transplantation) is widely used as radical therapy for various hematological diseases, an acute graft-versus-host disease (GVHD) is one of serious complications comparable to recurrence and infection. An adrenocortical steroid hormone (steroid) is a drug used for initial treatment (primary treatment) of this GVHD but its effect can be confirmed in only half of the cases (Blood. 2007; 109(10):4119-4126. (Non-patent document 1), and no secondary treatment has been established.

While enterobacteria and metabolites thereof are widely known to play important roles in suppression of inflammation and immunoregulation in a gastrointestinal tract, they are recently suggested of their potential in fecal microbiota transplantation (FMT) (Blood. 2014; 24(7):1174-1182. (Non-patent document 2)). With respect to FMT, inventions described in Japanese Unexamined Patent Application Publication (Translation of PCT) No. 2013-537531 (Patent document 1) and Japanese Unexamined Patent Application Publication (Translation of PCT) No. 2016-501852 (Patent document 2) are known.

However, relevance between a GVHD and FMT is unclear.

### PRIOR ART DOCUMENTS

### Patent Documents

Patent document 1: Japanese Patent Application Publication No. 2013-537531

Patent document 2: Japanese Patent Application Publication No. 2016-501852

### Non-Patent Documents

Non-patent document 1: Blood. 2007; 109(10):4119-4126.

Non-patent document 2: Blood. 2014; 124(7):1174-1182.

### SUMMARY OF INVENTION

### Problem to be Solved by Invention

The present invention has an objective of providing a composition for preventing or treating, in particular, an acute gastrointestinal tract graft-versus-host disease.

### Means for Solving Problem

The present inventors have gone through extensive investigation to solve the above-described problem, and as a result of which succeeded in preventing or treating a graft-versus-host disease by transplanting a composition comprising a fecal microbiota, thereby accomplishing the present invention.

### MODE FOR CARRYING OUT THE INVENTION

Thus, the present invention is as follows.

(1) A composition for preventing or treating a graft-versus-host disease, the composition comprising a fecal microbiota.

(2) The composition according to (1), wherein the fecal microbiota is contained in feces or a processed material thereof.

(3) The composition according to either one of (1) and (2), wherein the microorganism is a microorganism that belongs to any one genus selected from the group consisting of Lactobacillus, Bacteroides, Bifidobacterium, Faecalibacterium, Blautia and Clostridium, or a combination thereof.

(4) The composition according to any one of (1)-(3), wherein the graft-versus-host disease is a gastrointestinal acute graft-versus-host disease.

(5) The composition according to any one of (1)-(4), wherein the graft-versus-host disease is a steroid-resistant or steroid-dependent graft-versus-host disease.

(6) The composition according to any one of (1)-(5), which is in a form of capsule.

(7) A capsule formulation for preventing or treating a graft-versus-host disease, the formulation comprising the composition according to any one of (1)-(5).

(8) A method for treating a graft-versus-host disease, comprising the step of administering the composition according to any one of (1)-(6) or the capsule formulation according to (7) to a patient with the graft-versus-host disease.

(9) A method for preventing a graft-versus-host disease, comprising the step of administering the composition according to any one of (1)-(6) or the capsule formulation according to (7) to a patient targeted for hematopoietic stem cell transplantation before, after or both before and after said transplantation.

According to the present invention, a gastrointestinal acute graft-versus-host disease can be prevented or treated.

### BRIEF DESCRIPTION OF THE DRAWINGS

FIG. 1A Diagrams showing compositions of enterobacteria and results from immunoanalyses. Each panel shows change in the composition of the enterobacteria with time for each case.

(i) Case 1, (ii) Case 2, (iii) Case 3 and (iv) Case 4

FIG. 1B Diagrams showing compositions of enterobacteria and results from immunoanalyses.

Panel i) shows subpopulations of regulatory T cells (Treg). Treg can be classified into three subpopulations according to the expression levels of FoxP3 and CD45RA. FoxP3^{lo}CD45RA⁺ T cells (fraction 1) are classified as naive Treg cells, which differentiate into effector Treg (eTreg) under antigen stimulation.

FoxP3^{hi}CD45RA⁻ T cells (fraction 2) are classified as eTreg, which has a strong suppressive activity in the form of terminally differentiated cells. FoxP3^{lo}CD45RA⁻ T cells are classified as non-Treg (fraction 3), which has no suppressive activity that is characteristic of Treg and which secretes inflammatory cytokine. Panel ii) shows behaviors of the eTreg values (red line) and the eTreg/CD8⁺ T-cells (effector T cells) ratio (green line) in the peripheral blood for each case.

FIG. 2 A diagram showing the effect of steroid reduction. Cases 1-3 that reached CR succeeded in an average of 69% steroid reduction on Day 28 from the final FMT.

FIG. 3 Diagrams showing changes in the number of operational taxonomic units (OTUs) and the diversity index with time for each case.

A) Case 1, B) Case 2, C) Case 3 and D) Case4.

FIG. 4 Diagrams showing the number of FoxP3⁺CD4⁺ T cells (red line) and the FoxP3⁺CD4⁺ T cells/CD8⁺ T cells (effector T cells) ratio (blue line) in the peripheral blood for each case.

FIG. 5 Diagrams showing schedule and progress of the treatment, and results from the analysis of the enterobacterial flora for Case 5.

FIG. 6 Diagrams showing schedule and progress of the treatment, and results from the analysis of the enterobacterial flora for Case 6.

FIG. 7 Diagrams showing results from Unifrac analyses.

FIG. 8 Diagrams showing results from Unifrac analyses.

FIG. 9 Diagrams showing the results from analyzing the number of OTUs (operational taxonomic units).

FIG. 10 Diagrams showing schedule and progress of the treatments for Cases 7 and 8.

FIG. 11 A diagram showing schedule and progress of the treatment for Case 9.

FIG. 12 Diagrams showing schedule and progress of the treatments for Cases 10 and 11.

FIG. 13 A diagram showing schedule and progress of the treatment for Case 12.

### DETAILED DESCRIPTION OF THE INVENTION

The present invention relates to a composition for a graft-versus-host disease, comprising a fecal microbiota.

Herein, the abbreviations stand for the following terms.

FMT, fecal microbiota transplantation; mPSL, methylprednisolone; PSL, prednisolone; FK, tacrolimus; TAZ/PIPC, tazobactam/piperacillin; CFPM, cefepime; VCM, vancomycin; ST, sulfamethoxazole/trimethoprim; LVFX, levofloxacin; CAZ, ceftazidime; TEIC, teicoplanin; MEPM, meropenem; Fr, fraction; OTU, operational taxonomic unit.

Based on the thought that intervention in the enterobacterial flora with feces could lead to a novel prophylactic or therapeutic method for GVHD, the present inventors have attempted a fecal microbiota transplantation method (FMT) for GVHD. An FMT method is a therapeutic method in which a fecal suspension of a healthy subject is placed in a gastrointestinal tract to deliver a large amount of a normal bacterial flora, and which has been attempted for diseases that are considered to be associated with dysbiosis.

Since a fecal microbiota is contained in feces itself or in a processed fecal material, feces itself or a processed fecal material can be used as a composition of the present invention. The processed fecal materials include a suspension obtained by suspending collected feces into suitable aqueous liquid (for example, physiological saline, buffer, etc.), a suspension obtained by filtrating said suspension through a suitable sieve, gauze, filter or the like (for example, with a pore size of 0.1 mm-0.5 mm), or a precipitate obtained by centrifugation or the like. The processed material may also be obtained by freezing these compositions in a freezer or with liquid nitrogen, freeze-dried, or spray-dried. If aqueous liquid is to be used to obtain a suspension, the liquid of 1.5-3.0 ml can be used to suspend 1 g of feces. If a suspension is to be prepared and then bacteria are to be extracted therefrom by centrifugation and used, the liquid of 0.5-0.6 mL is used to resuspend 1 g of the bacteria.

A cryoprotectant and/or a lyoprotectant such as a sugar (sucrose, fructose, lactose, mannitol, etc.), glycerol, polyethylene glycol (PEG), trehalose, glycine, glucose, dextran, erythritol or the like may be added upon freezing or freeze drying.

According to the present invention, the collected feces or the processed material thereof can be stored for 6-10 hours after collecting or processing the feces. Although the storage temperature is not particularly limited, it is preferably at a temperature for refrigeration (for example, 4° C.).

The composition prepared as described above is used as the FMT material. The prepared FMT material is preferably stored under an anaerobic condition (for example, in an anaerobic unit, an anaerobic bag, etc.) until use. In this case, again, the storage temperature is preferably at a temperature for refrigeration (for example, 4° C.).

The composition of the present invention contains, as so-called beneficial bacteria, for example, a microorganism belonging to Lactobacillus, Bacteroides, Bifidobacterium or Faecalibacterium, or a combination of these microorganisms.

Therefore, when the composition of the present invention is transplanted, the above-mentioned microbiota will be dominant, for example, over microorganisms belonging to Corynebacterium and Streptococcus as well as Escherichia coli.

Furthermore, in order to ease the transplantation or in order to allow long-term use, for example, the composition of the present invention may take any form of powder, solid or liquid, and further, these powder, solid or liquid form can be made into a capsule formulation. A capsule formulation is advantageous in that complications such as bleeding caused by insertion of a tube, a large intestine fiberscope or the like can be avoided, and burden placed on the patient upon the transplantation can be reduced.

The composition of the present invention may further contain at least one selected from the group consisting of pH stabilizers, acidulants, antiseptics, vitamins, minerals, nutrition supplements, prebiotics and probiotics.

According to the present invention, in order to prevent the patient from developing other disease or infection, selection of donor feces is important. Therefore, according to the type of the donor from whom feces is collected and the recipient to be transplanted with the microbiota (for example, human or animal), the feces collected from the donor is preferably screened, for example, for the presence of at least one selected from the group consisting of retrovirus (for example, human immunodeficiency virus), hepatitis virus (hepatitis viruses A, B and C), syphilis, cytomegalovirus, Epstein-Barr virus and parasites.

According to the present invention, transplantation of a composition (unprocessed or processed fecal material) of the present invention containing a fecal bacterial flora is carried out between different individuals, for example, between humans or between animals. The composition of the present invention can be returned and transplanted to the same individual as the individual from which the composition was collected, or a fecal bacterial flora collected from one individual can be transplanted to other individual.

A disease targeted for the use of the composition of the present invention is a GVHD, including GVHD caused by hematopoietic stem cell transplantation. An example of GVHD includes, but not limited to, a gastrointestinal acute GVHD.

The transplantation method may be carried out either by oral administration or parenteral administration, and not particularly limited. For example, transplantation via a gastroduodenal tube, oral use using a capsule or the like filled with the composition, or transplantation into the large intestine using a large intestine fiberscope or through high-pressure enema can be employed.

The transplanted amount per single time is 150 ml-300 ml once a day in a case of a liquid form. If transplantation should be repeated depending on the state of the recipient, it is done every four days to two weeks for a total of 2-4 times.

Accordingly, the composition of the present invention can be transplanted (administered) to a GVHD patient to prevent or treat GVHD. In addition, the composition of the present invention can be administered before, after or both before and after hematopoietic stem cell transplantation to the patient receiving said transplantation to prevent GVHD.

In the context of “treatment” as used herein, the degree of suppression is not limited as long as development of GVHD can be suppressed. Therefore, “treatment” include both complete response (CR) and partial response (PR). Complete response (CR) means that all of the GVHD symptoms have improved while partial response (PR) means that improvement of stage 1 or more can be found. According to the present invention, the treatment is found effective (treated) if PR or CR is reached in a steroid-resistant case or if steroid reduction of 40% or more as compared to that before the treatment is succeeded in a steroid-dependent case.

Moreover, “prevention” means that GVHD development is suppressed in advance or the already occurred GVHD state does not get any worse.

### EXAMPLES

Hereinafter, the present invention will be described more specifically by way of examples. The scope of the present invention, however, should not be limited to these examples.

### Example 1

[Subjects and Methods]

FMT Subjects

FMT was conducted for 4 cases of steroid-resistant (3 cases) and steroid-dependent (1 case) gastrointestinal acute GVHD (Table 1). Other than the gastrointestinal tract, skin and a liver can also be target organs of an acute GVHD, but this time only the gastrointestinal tract was targeted since most of the basic researches on enterobacteria involved evaluation of immune response of the local gastrointestinal tracts, and since a gastrointestinal acute GVHD is involved in most of the fatal GVHD as compared to other organs. A steroid-resistant case refers to a case where the state did not change even more than 5 days after the start of the treatment with an adequate amount of steroid (1 mg or more prednisolone per weight of the patient) while a steroid-dependent case refers to a case where it was once responsive to a steroid treatment but worsened with the reduction thereof and thus needed to increase the amount again (case having difficulty with reduction).

As to the additional cases, the target criteria for Cases 10 and 11 were the same as described above. Meanwhile, for Cases 5-9, in addition to the above-described criteria, a case of a gastrointestinal acute GVHD that worsened 3 days after the start of the steroid treatment were also included. Case 12 was a refractory case where none of an increase in steroid, a steroid pulse therapy and anti-thymocyte globulin (ATG) was effective.

Even a case where a patient was also suffering from other gastrointestinal tract lesion was included, if a gastrointestinal acute GVHD was considered as the main cause of diarrhea.

Exclusion criteria were as follows.

1) GVHD that responded to steroid treatment

2) GVHD that worsened after the primary treatment with steroid (regardless of the affected organ)

3) Case having uncontrollable infection

4) When diarrhea seemed to result from a cause other than GVHD

With respect to Criterion 2), exacerbation of the gastrointestinal acute GVHD was excluded for Cases 5-9 and 12 among the additional cases.

Selection of FMT Donors

A donor candidate was selected from the spouse or relatives within the second degree of kinship of the patient.

The candidate was selected from the age in the range of 20-64 who has none of the following infection risks.

1) Got new tattoos or pierced within the last three months

2) Had sexual contact with new partner within the last three months

3) Received a blood transfusion within the last three months

4) Had travel history to the tropical regions within the last three months

5) Used any antibiotics within the last one month

6) Had previous history of malignant disease or inflammatory bowel disease

7) Had any digestive organ symptoms such as diarrhea on the day of FMT (as confirmed on the day of FMT)

If the donor candidate had no problem with the above-listed items, blood was taken and feces was examined. Blood testing was conducted for checking HIV, human T-lymphotropic virus type I (HTLV-1), hepatitis A, B and C, syphilis, cytomegalovirus (CMV) and Epstein-Barr virus (EBV), and feces was examined to check parasites, Clostridium difficile, Cryptosporidium, Giardia, Microsporidia, Entamoeba hislolytica, Cyclospora, Isospora, Dientamoeba fragilis, Blastocystis hominis, Schistosoma, pathogenic bacteria and the like. A patient having a previously infected pattern of CMV or EBV was judged to have no problem.

FMT Method

On the day of performing FMT, feces was collected from the donor and stored at 4° C. in an anaerobic state until use.

The feces was adjusted upon inserting a gastroduodenal tube into the patient. The feces was adjusted by first weighing the feces, then adding 200-300 mL of sterile physiological saline according to the weight and thoroughly agitating the resultant until it became uniform. The resultant was filtrated through a metal sieve once to remove large undigested matters. Subsequently, the resultant was filtrated twice with sterile gauze to prepare a suspension. Once both were ready, FMT was conducted.

A 50-mL syringe was filled with the suspension, and the fecal suspension was administered via a gastroduodenal tube. The administration rate was set not to exceed 50 mL per 30 seconds. Once the entire suspension was administered, 50 mL of physiological saline was used to wash inside the tube and the tube was withdrawn to complete the procedure.

Unless an FMT-related adverse event of grade 3 or higher was observed, additional administration was allowed once in 4-14 days while checking the effect. In this case, the feces was collected from the same donor as the initial donor. The feces transplantation was carried out within 12 hours, if possible within 8 hours, from feces collection from the donor.

As to patients fasting due to the gastrointestinal acute GVHD, supplements containing dietary fiber/oligosaccharide (GFO (registered trademark): Otsuka Pharmaceutical Factory, Inc.) was given as prebiotics from the day before the first day of FMT. Moreover, antibiotics were stopped as much as possible for 24 hours before and after FMT.

Safety was judged by any adverse events that newly occurred within a week after FMT or deterioration of grade 1 or more, where adverse events were evaluated according to the National Cancer Institute Common Terminology Criteria for Adverse Events (CTC-AE) version 4.0.

The therapeutic effects were evaluated 4 weeks after the last FMT, and as the maximum response before the rituxan administration for Case 4 since rituximab (anti-CD20 antibody) was used for EBV reactivation during the progress in this case.

The therapeutic effects were judged according to the following criteria.

1) Complete response (CR): Amelioration of all gastrointestinal tract symptoms

2) Partial response (PR): Amelioration of stage 1 or more

3) Progression (PG): Deteriorative progression

4) No change (NC): No significant amelioration

FMT was judged to be effective when PR or CR was reached for the steroid-resistant cases, and when reduction of 40% or more was succeeded as compared to the level prior to the therapy for the steroid-dependent case.

Analysis of Enterobacterial Flora (Meta-16S Analysis Method)

Each of the feces of the donor, the adjusted feces liquid prepared and the feces of the patient (before FMT, a day after each FMT, and 1, 2 and 4 weeks after the last FMT) was partially used to conduct analysis of the enterobacterial flora.

DNAs of the bacteria were extracted from each sample, which were amplified by PCR (polymerase chain reaction) using primers designed to cover the variable regions V1-V2 of 16S rRNA gene, and then sequenced. The resulting sequence was subjected to quality checking and 3,000 sequence data reads that passed were used for the analysis. Operational taxonomic units (OTUs) clustering was performed by employing UCLUST algorithm and a threshold of 96% homology. Furthermore, the bacterial species were identified by checking against the existing database. Moreover, the Shannon index was used for alpha diversity evaluation.

Immunoanalysis (Flow Cytometry Method)

Peripheral blood was collected from the patient before FMT and about a week after each FMT. Mononuclear cells were isolated from these blood to evaluate immunodynamics by a flow cytometry method. Antibodies used for the evaluation were as follows.

Alexa Fluor 700 (AF700)-conjugated anti-CD3 (UCHT1) mAb, V500-conjugated anti-CD4 (RPA-T8) mAb (which were purchased from BD Biosciences), eFluor780 fixable viability dye (which was purchased from Affymetrix eBioscience), peridinin-chlorophyll-protein complex-cyanine 5.5 (PerCP-Cy5.5)-conjugated anti-CD194 (CCR4) mAb, Fluorescein isothiocyanate (FITC)-conjugated anti-CD127 (IL-7Rα) (A019D5) mAb, Allophycocyanin (APC)-conjugated anti-CD152 (CTLA-4) (L3D10) mAb, Brilliant Violet (BV) 605-conjugated anti-CD197 (CCR7) mAb, BV711-conjugated anti-CD45RA (HI100) mAb, BV785-conjugated anti-CD8 (RPA-T8) mAb, BV42-conjugated anti-CD279 (PD-1) (EH12.2H7) mAb, PE-Cy7-conjugated anti-CD152 (CTLA-4) (L3D10) mAb and PE/Dazzle 594 anti-CD25 (M-A251) mAb (which were purchased from BioLegend).

Intracellular FoxP3 staining was carried out with R-phycoerythrin (PE)-conjugated anti-FoxP3 (236A/E7) mAb (Affymetrix eBioscience) after fixing with Foxp3/Transcription Factor Staining Buffer Set (Affymetrix eBioscience).

The stained cells were washed, examined with LSR Fortessa (BD Biosciences) and analyzed utilizing FlowJo version 10.0.8 software (FlowJo, Ashland, Oreg.).

[Results and Discussions]

All of the four targeted cases suffered from acute myeloid leukemia (AML) (Table 1). Case 3 was diagnosed as a late-onset acute gastrointestinal tract GVHD. Initial FMT was carried out 92 days after the transplantation (Day 92), and 1 mg/kg or more of steroid in terms of methylprednisolone was administrated upon conducting FMT in all cases. In addition, they had some sort of infectious complications (Table 1).

TABLE 1
Patient characteristics, adverse events and response

| | Case 1 | Case 2 | Case 3 | Case 4 |
|---|---|---|---|---|
| Age, y/Gender | 64/female | 44/female | 48/male | 42/male |
| Diagnosis | AML | AML with 3q26.2 abn | MK-AML | AML-MRC |
| Indication for FMT | Resistant | Resistant | Dependent | Resistant |
| GVHD stage (overall) | | | | |
| Gut | 1 | 4 | 1 | 2* |
| Skin | 0 | 0 | 0 | 0 |
| Liver | 0 | 3 | 3 | 1 |
| GVHD grade (overall) | II | III | II | IV^{†} |
| GVHD stage at start of FMT | | | | |
| Gut | 1 | 4 | 1 | 2* |
| Skin | 0 | 0 | 0 | 0 |
| Liver | 0 | 0 | 0 | 1 |
| GVHD grade at start of FMT | II | III | II | IV^{†} |
| Intinal treatment dose of steroid | 2 mg/kg of mPSL | 2 mg/kg of mPSL | >2 mg/kg of mPSL | 1-2 mg/kg of mPSL |
| Dose of steroid at start of FMT | 1 mg/kg of mPSL | 1 mg/kg of mPSL | >2 mg/kg of mPSL | 2 mg/kg of mPSL |
| Treatment for GVHD other than | FK, beclomethasone | FK, beclomethasone, | FK, beclomethasone | FK, beclomethasone, |
| systemic steroid | | octreotide, loperamide, | | octreotide |
| | | fentanyl | | |
| Infectious complications and | | | | |
| treatment at start of FMT | | | | |
| Clostridium difficile toxin | — | — | — | — |
| Comorbid infection | CMV antigenemia | IPA | IPA | Sepsis (catheter |
| | | CMV retinitis | | infection) |
| | | | | CMV enteritis |
| Antibiotics | ST, TAZ/PIPC | LVFX | CFPM + VCM | CFPM |
| Cessation of antibiotics | Yes (TAZ/PIPC) | Yes | Yes | No |
| Antifungals | MCFG | VRCZ | L-AmphB | MCFG |
| Antivirals | Foscarnet | Ganciclovir (intraocular) | Aciclovir | Foscarnet |
| | | Foscarnet | | |
| Adverse events (grade) | | | | |
| 1st FMT | Abdominal pain (1) | Belch (1) | Diarrhea (1) | Hypoxia (2) |
| | | Pharyngolaryngeal pain (1) | Anemia (2→3) ^{□} | Delirium (1) |
| | | Diarrhea (2) | Thrombocytopenia | Lower GI |
| | | Hypokalemia (L-AmphB | (3→4) ^{□} | hemorrhage (1) |
| | | induced) (2) | | Hypothyroidism (1) |
| | | | | γGTP↑ (1→2) |
| | | | | Abdominal pain (1) |
| | | | | Fever (1) ^{□} |
| 2nd FMT | Abdominal pain (1) | Abdominal pain (1) | NA | Blood bilirubin increased |
| | Pharyngolaryngeal pain (1) | Pharyngolaryngeal pain (1) | | (1→3) |
| | Nausea (1) | Diarrhea (2) | | γGTP↑ (2→3) |
| | | | | PAF (1) |
| | | | | TA-TMA (2)^{§} |
| Response‡ | Complete response | Complete response | Complete response | Partial response |

Abbreviations: FMT, fecal microbiota transplantation; GVHD, graft-versus-host disease; AML, acute myeloid leukemia; AML with 3q26.2 abn, AML with 3q26 abnormality, MK-AML, AML with monosomal karyotype; AML-MRC, AML with myelodysplasia-related changes; mPSL, methylprednisolone; FK, tacrolimus; CMV, cytomegalovirus; IPA, invasive pulmonary aspergillosis; ST, sulfamethoxazole/trimethoprim; TAZ/PIPC, tazobactam/piperacillin; LVFX, levofloxacin; CFPM, cefepime; VCM, vancomycin; MCFG, micafungin; VRCZ, voriconazole; L-AmphB, liposomal amphotericin B; GI, gastrointestinal; PAF, paroxysmal atrial fibrillation; TA-TMA, transplant-associated thrombotic microangiopathy; NA, not applicable.
*Downgraded one stage because of CMV enteritis
^{†}Graded as IV because of extremely poor performance status
‡Response of FMT was evaluated 28 days after last infusion (Cases 1-3) or as maximum response before rituximab administration (Case 4)
^{§}TA-TMA was graded using the common toxicity criteria proposed by Ho et al. (2005)
^{□} Recovered in 1 day

The median feces volume used for FMT was 126 g (34-307 g) while the amount of the resulting adjusted feces liquid was 180-230 mL. The adjusted feces liquid was administered by spending 4-8 minutes. The median time that took from the feces collection to FMT was 6 hours (Table 2).

TABLE 2
Patient's characteristics and information regarding fecal microbiota transplantation

| | | Case 1 | Case 2 | Case 3 | Case 4 |
|---|---|---|---|---|---|
| Disease status at SCT | | CR | Refractory relapse | Primary refractory | non-CR |
| | | | after 1st SCT | | |
| Conditioning regimen | | Flu/Bu(X8)/ | Flu/Mel/AC/ATG/ | Flu/Bu(X16)/ | Flu/AC/CY/ATG |
| | | 4 Gy TBI | 4 Gy TBI | 4 Gy TBI | 8 Gy TBI |
| Donor type | | MUD | HFD | MMRD | HFD |
| Donor source | | Bone marrow | Peripheral blood | Peripheral blood | Peripheral blood |
| GVHD prophylaxis | | FK + sMTX | FK + mPSL + ATG | FK + MMF | FK + mPSL + ATG |
| FMT donor | | Husband | Sister | Wife | Wife |
| Patient's body weight at FMT (kg) | 1st | 38.1 | 46.3 | 45.1 | 94.1 |
| | 2nd | 39.1 | 43.3 | NA | 93.1 |
| Feces volume used for FMT (g) | 1st | 34 | 117 | 186 | 126 |
| | 2nd | 307 | 53 | NA | 143 |
| Time from feces collection to FMT (h) | 1st | 4 | 3.25 | 9 | 8.5 |
| | 2nd | 6 | 2.75 | NA | 8.5 |

Abbreviations: SCT, stem cell transplantation; GVHD, graft-versus-host disease; FMT, fecal microbiota transplantation; CR, complete remission; Flu, fludarabine. Bu, busulfan; TBI, total body irradiation; Mel, melphalan; AC, cytosine arabinoside; ATG, antithymocyte globulin; CY, cyclophosphamide; MUD, matched unrelated donor; HFD; haploidentical family donor; MMRD, mismatched related donor; FK, tacrolimus; sMTX, short course of methotrexate; mPSL, methylprednisolone; MMF, mycophenolate mofetil; NA, not applicable

Adverse events that were considered to be obviously involved with FMT were mild and temporary (as underlined in Table 1). Case 4 developed various complications including hypoxemia, paroxysmal atrial fibrillation, lower gastrointestinal bleeding, cholestatic liver diseases and else. However, since bleeding occurred at lower gastrointestinal tract and atrial fibrillation occurred 4 days after FMT, FMT did not seem to be the direct cause. Rather, very serious fundamental general conditions (deterioration of the performance status, significant hypoalbuminemia, severe cytopenia that requires regular blood transfusion, use of various drugs, EBV reactivation, etc.) appeared likely to be the causes of these complications.

In Case 4, the patient got fever 2 days after the second FMT but focus of infection was not identified and fever abated a day after changing the antibiotic.

Consequently, it was considered that FMT can be administered relatively safely even in a case where immunity had severely deteriorated immediately after the transplantation.

As to the therapeutic effect, all of the cases were responsive with 3 CR cases and 1 PR case (max. response). In particular, amelioration of the symptoms was seen in a few days to about a week in the steroid-resistant case. Moreover, in the CR cases (Cases 1-3), steroid was able to be reduced by an average of 69% 28 days after the final administration (FIG. 2).

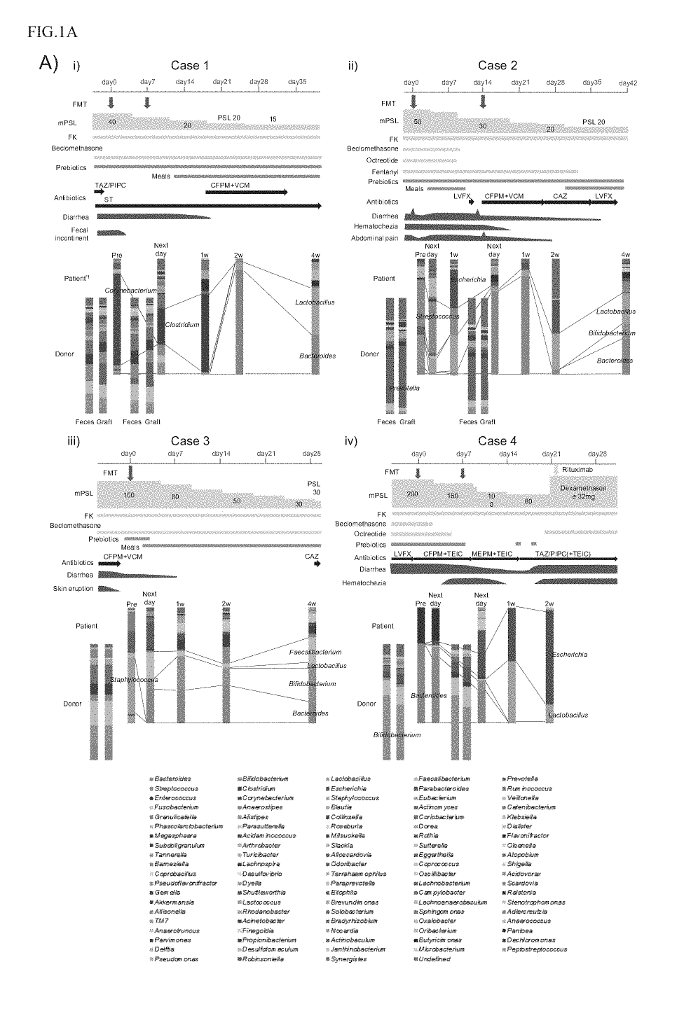

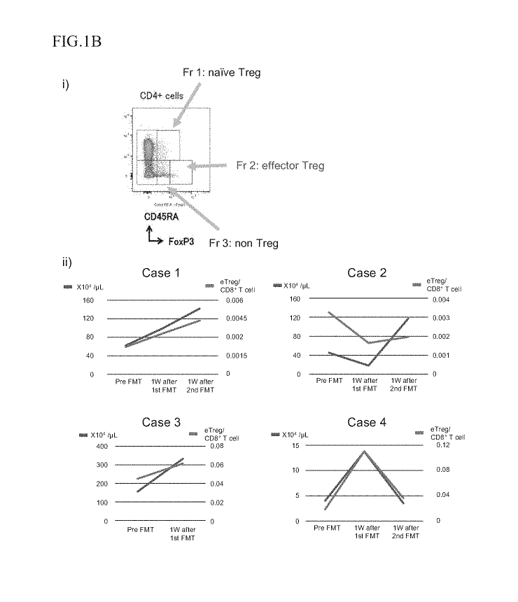

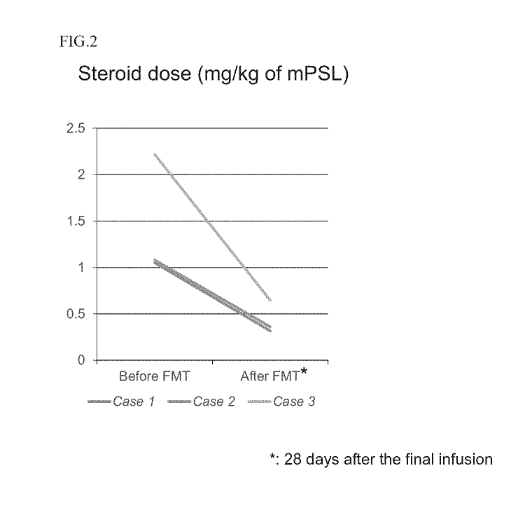

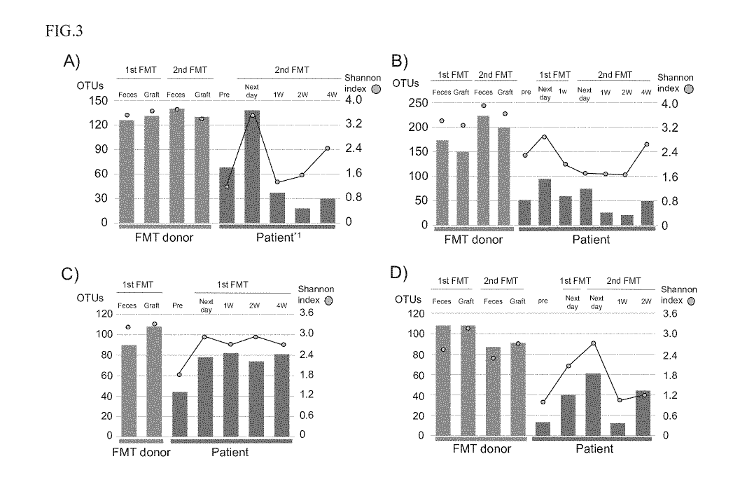

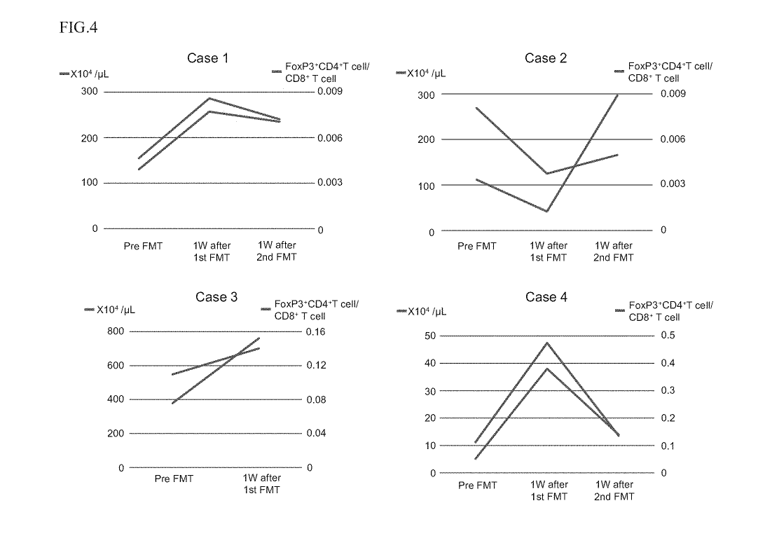

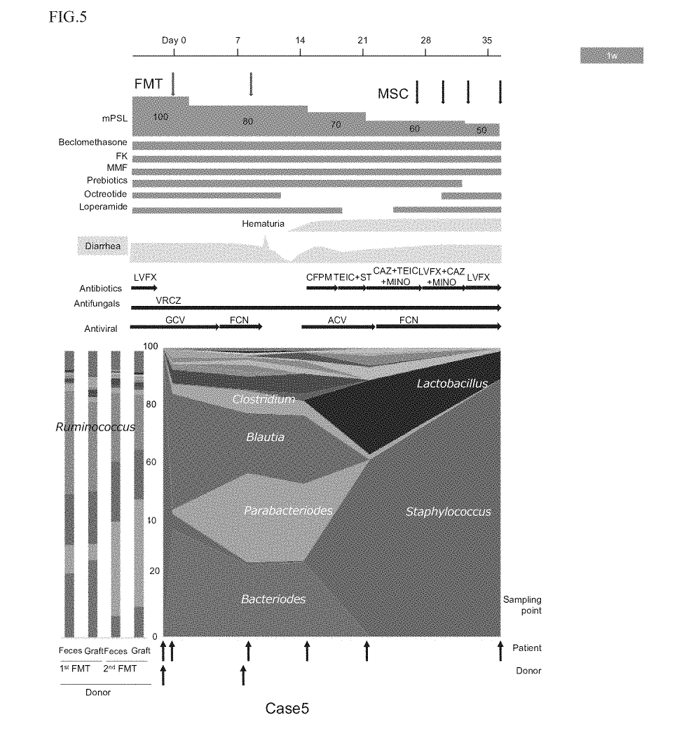

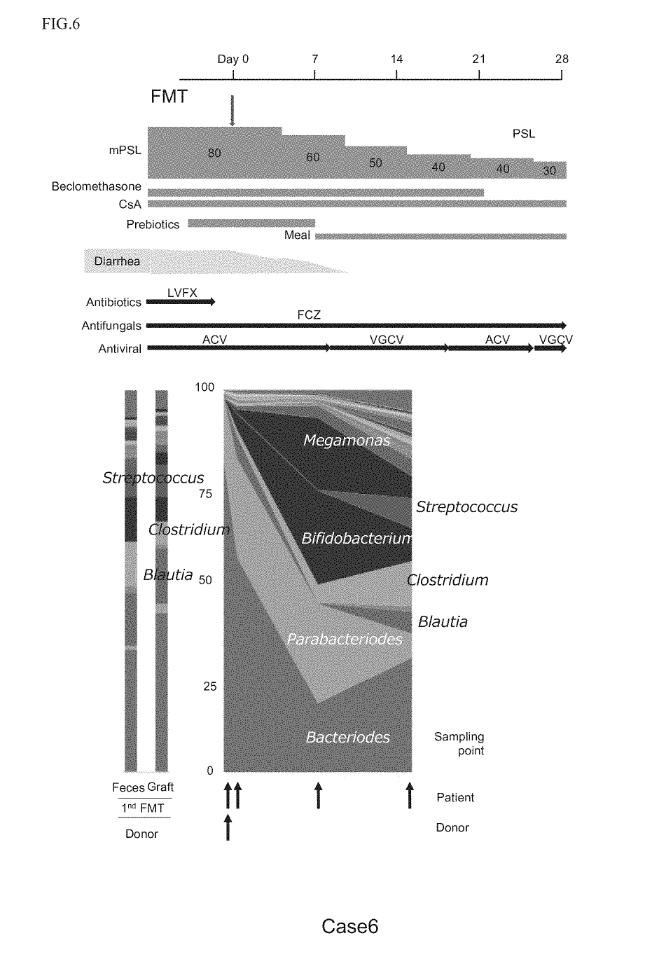

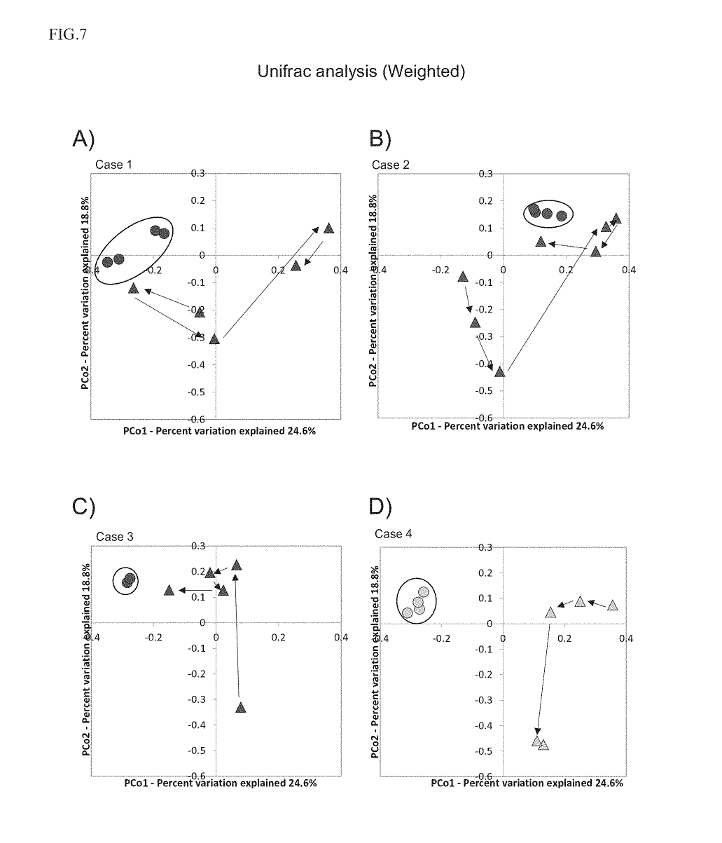

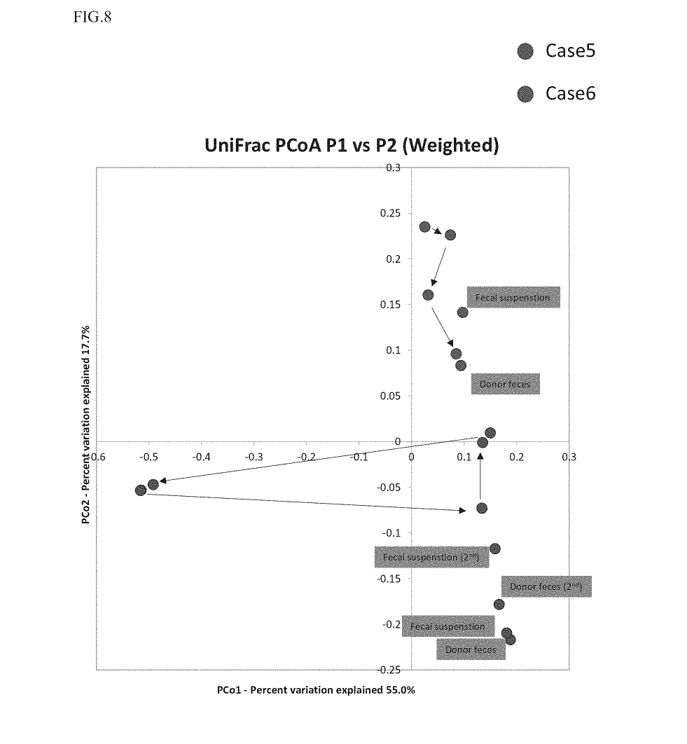

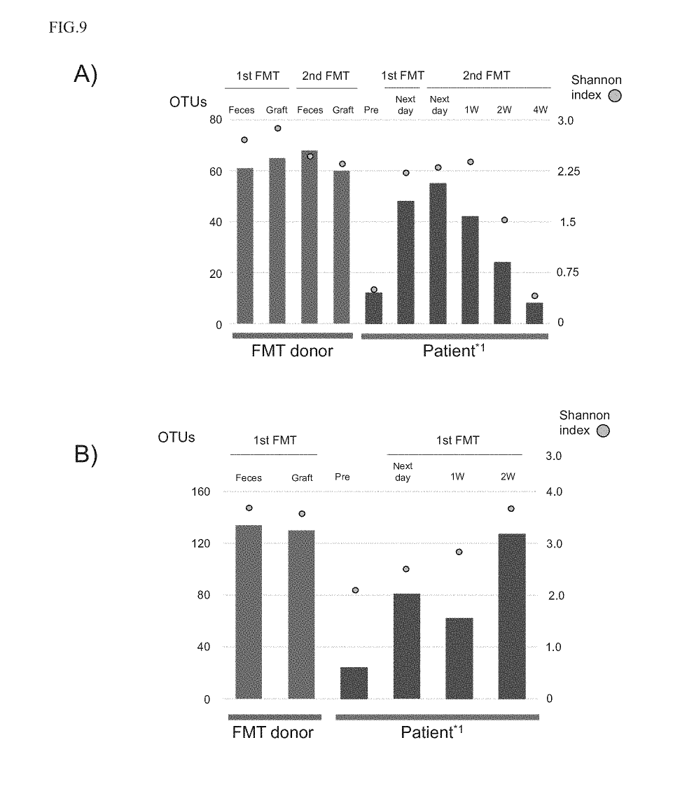

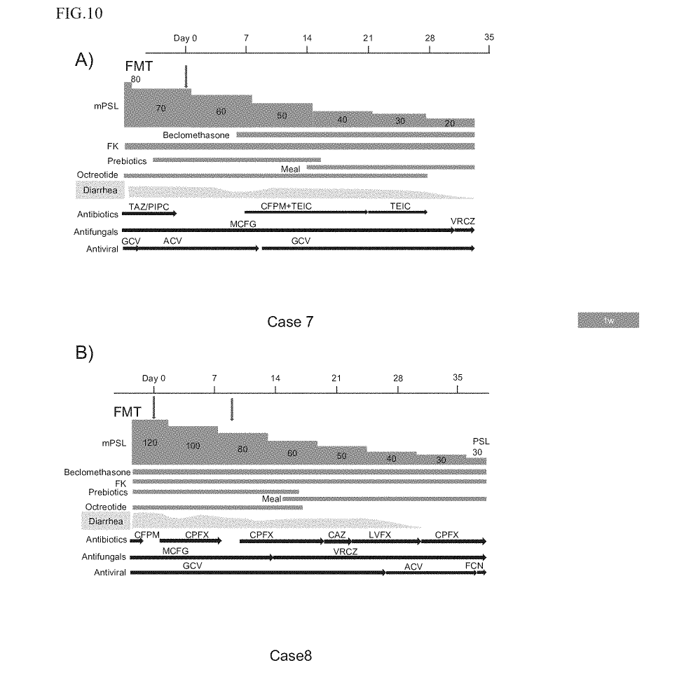

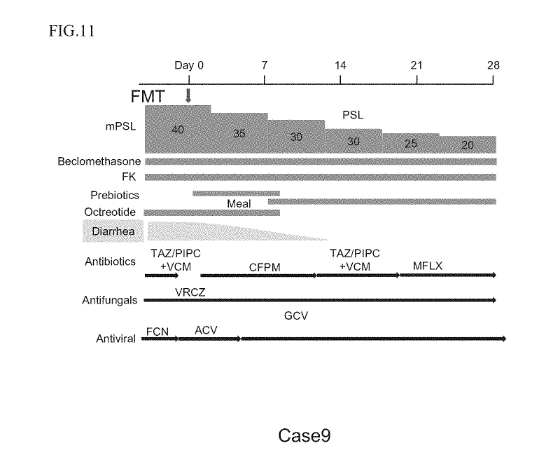

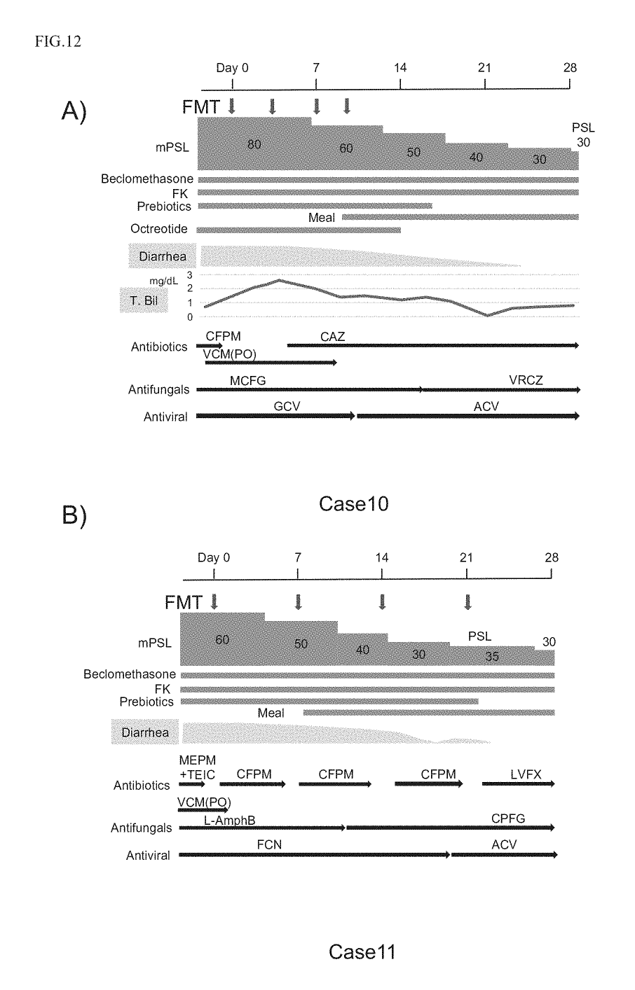

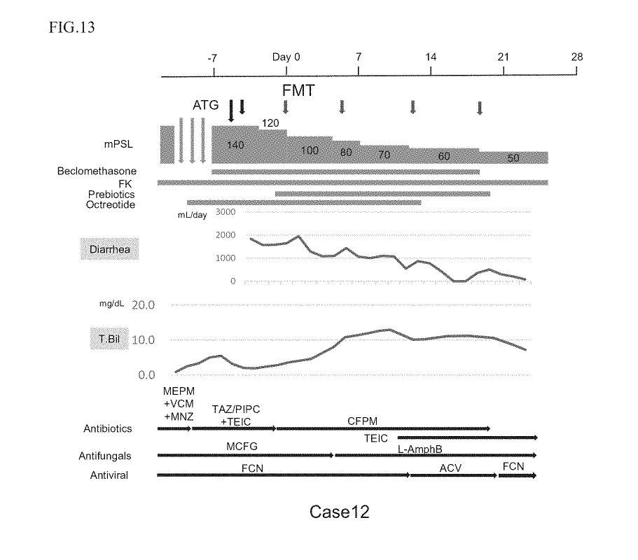

Change in the fecal bacterial flora was as shown in FIG. 1A. Case 1 showed a relatively steady progress after FMT. In Case 1, Corynebacterium was dominant prior to FMT but Lactobacillus and Bacteroides predominated in the end after FMT (FIG. 1A, panel i)). In Case 2, although the effect of the first FMT was limited, the effect gradually improved after the second FMT. In Case 2, Streptococcus was dominant prior to FMT, which remained after the first FMT but disappeared upon the second FMT and Lactobacillus, Bacteroides and Bifidobacterium took over in the end (FIG. 1A, panel ii)).

Case 3 was a steroid-dependent case where diarrhea was ameliorated by increasing the steroid again, and remission was maintained thereafter. In Case 3, Staphylococcus was dominant prior to FMT but the composition of the feces largely changed on the day after FMT where Bacteroides, Lactobacillus, Bifidobacterium, Faecalibacterium and the like predominated (FIG. 1A, panel iii)). In Case 4, although the amount of diarrhea temporarily decreased after two courses of FMT, transplant-associated thrombotic microangiopathy (TA-TMA) occurred probably due to the immunosuppressant. Therefore, the immunosuppressant was rapidly decreased, by which GVHD relapsed. In Case 4, Bifidobacterium slightly increased after two courses of FMT but eventually Escherichia accounted for a large proportion (FIG. 1A, panel iv).

From the above results, the composition of enterobacteria of each case appeared to well correlate with the clinical progress after FMT. Specifically, while Corynebacterium, Staphylococcus, Streptococcus and the like, that do not normally predominate, accounted for a large proportion prior to FMT, normally predominant symbiotic bacteria and so-called beneficial bacteria including Bacteroides, Lactobacillus, Bifidobacterium and Faecalibacterium increased as diarrhea ameliorated after FMT. On the other hand, in the relapsing case (Case 4), Escherichia that normally does not predominate proliferated in the end. This increase in Escherichia coincides with the results obtained in mouse GVHD models. In some cases, the number of OTUs and alpha diversity did not sufficiently recover (FIG. 3).

Amelioration of a gastrointestinal acute GVHD by FMT may not require complete diversity restoration. In fact, regardless of the development of GVHD, the number of OTUs is very small in most of the transplantation patients as compared to that in healthy subjects. Moreover, there were cases where the patients had to start taking antibiotics again during the proceeding of this research (Cases 1 and 2), but the research proceeded without relapse of GVHD. Use of the antibiotic that had a relatively weak spectrum against anaerobic bacteria seemed to be related to the survival of anaerobic bacteria and had influence on the outcome. Furthermore, in all of the cases, concurrent infection went on without getting worse. From these facts, FMT was considered to have small effect on the immunity to infection.

Regulatory T cells (Treg) play an important role in immunoregulation where they act to suppress immunity, and also play an important role for GVHD. Tregs are classified into three subpopulations by the expression levels of CD45RA and FoxP3 (FIG. 1B, panel i)), among which CD45RA⁻FoxP3^{hi} fraction (Fr2) is particularly classified as effector Treg (eTreg) and has strong inhibitory action. Behavior of eTreg in the peripheral blood was evaluated, where the results showed that eTreg increased during the phase where FMT was found to be effective. This tendency was almost the same in the eTreg/CD8⁺ T cell ratio (FIG. 1B, panel ii)).

Similar results were also found for CD4⁺FoxP3⁺ T cells as a whole (FIG. 4). This suggests the possibility of FMT also having an influence on the behavior of the systemic immunity. In fact, some of the previous reports show relevance between enterobacteria and acute GVHD itself, which is seemingly supported by the results of the present examples. Moreover, these facts also suggested that FMT was also effective for GVHD in other organs (skin/liver).

Thus, it was considered that the change (in a good way) in the composition of enterobacteria due to FMT may possibly have an anti-inflammatory action and may cure GVHD.

[Conclusion]

FMT was also able to be carried out for a patient immediately after transplantation. This could be another new therapeutic/prophylactic strategy for GVHD.

### Example 2

Analyses of Progress and Bacterial Flora of Additional Cases

In this example, progress and the bacterial flora were analyzed for the additional cases in the same manner as Example 1.

(1) Additional Data for Analyzing Progress and Bacterial Flora for Additional Cases (FIGS. 5-8)

Case 5: 62 years old, female. FMT was conducted twice for a gastrointestinal acute GVHD at stage 1 following umbilical cord transplantation for acute myeloid leukemia (AML) (FIG. 5). Feces temporarily normalized in 2-3 days after the second FMT but the patient concurrently developed hemorrhagic cystitis. This triggered worsening of the acute gastrointestinal tract GVHD again. Therefore, a treatment with bone-marrow-derived mesenchymal stem cells (MSC) was added, but no improvement was seen. Feces prior to FMT was mostly occupied by Staphylococcus. Following FMT, the composition of bacteria changed largely and the composition of feces became similar to that of the donor. Bacteroides, Parabacteroides, Blautia, Clostridium and else increased but returned to the state before FMT after the relapse. The results from the Unifrac analysis also confirmed this behavior (FIG. 8).

Case 6: 40 years old, female. FMT was conducted for a gastrotintestinal acute GVHD following living-relative transplantation for acute lymphoid leukemia. Feces normalized about 10 days after FMT. While the feces was mostly occupied by Bacteroides and Parabacteroides prior to the transplantation, various bacteria such as Blautia, Clostridium, Bifidobacterium, Megamonas, Streptococcus and the like increased following FMT. This was similar to the composition of the donor. In fact, similarly to the donor composition after the transplantation was also confirmed by Unifrac analysis (FIG. 8).

Among the 3 cases (Cases 1-3) that were confirmed to be effective by Unifrac analysis of the 4 cases described in Example 1, the compositions of Cases 2 and 3 were confirmed to be similar to those of the donors when a therapeutic effect could be observed (FIGS. 7A-D). In addition, the number of OTUs (operational taxonomic units) and diversity were improved in Cases 5 and 6 when a therapeutic effect could be observed (Case 5: FIG. 9A, Case 6: FIG. 9B).

As to the results from the Unifrac analyses and the change in diversity, changes such as becoming similar to the composition of the donor or improvement in diversity, in other words, improvements in enterobacteria, were seen after FMT, not in all, but in many of the cases that showed effective outcomes. This suggests that improvement of the enterobacteria contributed to the treatment. In Case 1, although the composition was rather unalike from the donor composition after FMT as evaluated by Unifrac analysis, the diversity was eventually improved (Shannon index: 1.2 to 2.4), suggesting that dysbiosis that may trigger inflammation was improved.

(2) Other Experimental Cases

Progress of the additional cases subjected to FMT are shown in FIGS. 10 and 11. Although relapse was seen after the treatment in some cases, effects such as amelioration of diarrhea or the like had been observed after FMT in most cases. Although Case 7 was judged to be unchanged on the evaluation day (28 days after the last FMT), it thereafter reached CR on Day 35. After all, FMT appears to have a certain level of effect on a gastrointestinal acute GVHD.

All of the cases including the 4 cases described in Example 1 and the cases described in (1) above are summarized in Table 3.

TABLE 3

| | | | | Gut aGVHD (stage) | | | Response | | | | | |
|---|---|---|---|---|---|---|---|---|---|---|---|---|
| | | | | | | at the | | at the | | | | |
| | | | | | at the | maximun | at the | maximun | | | | |
| | | | | | time | response | time | response | at the | Steroid dose | | |
| | | Under- | | at | point of | (1st FMT~ | point of | (1st FMT~ | final | | at the time | |
| | Age/ | lying | Route | 1st | eval- | evaluation | eval- | evaluation | follow- | | point of | reduction |
| Case | gender | disease | of FMT | FMT | uation*¹ | point) | uation*¹ | point) | up | at 1st FMT | evaluation*1 | rate (%) |
| 1 | 64/ | AML | ND | 1 | 0 | 0 | CR | CR | CR | 40 mg of mPSL | 15 mg of PSL | 70.0 |
| | F | | | | | | | | | | | |
| 2 | 44/ | AML | ND | 4 | 0 | 0 | CR | CR | CR | 50 mg of mPSL | 20 mf of PSL | 68.0 |
| | F | | | | | | | | | | | |
| 3 | 48/ | AML | ND | 1 | 0 | 0 | CR | CR | CR | 100 mg of mPSL | 30 mg of PSL | 76.0 |
| | M | | | | | | | | | | | |
| 4 | 42/ | AML | ND | 2 | — | 1 | —*3 | PR | PG | 200 mg of mPSL | — | — |
| | M | | | | | | | | | | | |
| 5 | 62/ | AML | ND | 1 | 1 | 0 | PG | CR | PG | 100 mg of mPSL | — | — |
| | F | | | | | | | | | | | |
| 6 | 40/ | Ph + ALL | ND | 1 | 0 | 0 | CR | CR | CR | 80 mg of mPSL | 30 mg of PSL | 70.0 |
| | F | | | | | | | | | | | |
| 7 | 41/ | CML-LBC | ND | 1 | 1 | 1 | NC | NC | CR | 70 mg of mPSL | 30 mg of mPSL | 57.1 |
| | F | | | | | | | | | | | |
| 8 | 60/ | AML | ND | 2 | 0 | 0 | CR | CR | PG | 120 mg of mPSL | 30 mg of PSL | 80.0 |
| | M | | | | | | | | | | | |
| 9 | 59/ | CMML | ND | 1 | 0 | 0 | CR | CR | CR | 40 mg of mPSL | 20 mg of PSL | 60.0 |
| | F | | | | | | | | | | | |
| 10 | 69/ | AML | Capsule | 1 | 0 | 0 | CR | CR | CR | 80 mg of mPSL | 30 mg of PSL | 70.0 |
| | F | | | | | | | | | | | |
| 11 | 25/ | Ph + ALL | Capsule | 3 | 0 | 0 | CR | CR | CR | 60 mg of mPSL | 30 mg of PSL | 60.0 |
| | M | | | | | | | | | | | |
| 12 | 30/ | AML | ND + | 3 | 1 | — | —*3 | PR | PR | 120 mg of mPSL | 50 mg of | 58.3 |
| | F | | Capsule | | | | | | | | mPSL*2 | |
| | | | | | | | | | | | Meean | 66.9 |
| | | | | | | | | | | | Median | 69 |

Abbreviations:
F, female;
M, male;
AML, acute myeloid leukemia;
Ph + ALL, Philadelphia chromosome positive acute lymphoblastic leukemia;
CML-BC, chronic myelogenous leukemia lymphoid blactic crisis;
CMML, chronic myelomonocytic leukemia;
ND, nasoduodenal tube;
FMT, fecal microbiota transplantation;
CR, complete remission;
PR, partial remission;
NC, no change;
PG, progression,
*¹28 days after the final FMT in ND cases (Case 1-9), and 28 days after the start of FMT in capsule cases (Case 10-12)
*2Reduction rate were evaluated by using final dose of steroid
*3These patients died before day 28

Considering that the response rates of secondary GVHD treatments are mostly about 40%-60%, these results seem to be promising. The cases with sustained effects succeeded in extensive steroid reduction within relatively short period, and thus the present invention also seemed to contribute greatly to avoid the infection risk caused by long-term immunosuppression (Table 4). The relapse may have been caused because the number of rounds of FMT was twice at most in our clinical tests.

(3) Effect of FMT on GVHD of Other Organs

Although only gastrointestinal tracts were targeted in this example, livers were also examined in Cases 10 and 12. As a result, GVHD in the livers was suspected and it improved by FMT.

In Case 10, FMT was carried out for a gastrointestinal acute GVHD after donor lymphocyte infusion. The total bilirubin (T.Bil) had increasing tendency around FMT, which increased to 2.6 mg/dL immediately before the second FMT but thereafter decreased to a normal value (FIG. 12A).

In Case 12, FMT was carried out for a refractory GVHD because steroid pulse therapy or anti-thymocyte globulin (ATG) did not have obvious effect. Diarrhea ameliorated after FMT. T.Bil temporarily improved after the steroid pulse therapy but thereafter increased up to 12.9 mg/dL. However, following that peak, T.Bil gradually decreased eventually to 7.3 mg/dL (FIG. 13).

Although other drugs cannot be completely denied as the cause of the T.Bil increase in the above-described 2 cases, there was no apparent suspicious drug and thus it was likely to be GVHD. Since there was a response to the steroid pulse therapy particularly in Case 12, it was very likely to be GVHD. Since they tended to decrease upon FMT, effectiveness of FMT was also suggested for acute GVHD in organs other than the gastrointestinal tract.

### Example 3

Effectiveness of FMT Using Capsule

Other than the transduodenal administration conducted so far, the present inventors also carried out FMT using a capsule. Cases 10 and 11 correspond to this where sufficient effect was likely observed by a method using a capsule (FIG. 12).

A method for preparing a capsule and a method for carrying out the same were as follows by revising previously reported methods^{1,2}.

1) A fecal suspension was prepared in the same manner as Example 1 and 50 mL each was divided into a tube.

2) Centrifugation was carried out at 6,000 g for 15 minutes. Following centrifugation, two layers of pellets were obtained. The lower layer were insoluble pellets mainly containing impurities, while the upper layer were pellets mainly consisting of the bacterial flora (partially collected to confirm by gram staining) which could easily be suspended in physiological saline. The upper layer was collected and suspended in Glyceol-containing physiological saline to prepare a bacterial solution. The final concentration of Glyceol was adjusted to be 10-20%.

3) 450 μL each of the solution prepared in 2) above was used to fill a #1 acid-resistant capsule (DRcaps (registered trademark): Capsgel), which was each placed in a cryotube and then placed in liquid nitrogen for rapid freezing. The prepared capsule was stored at −80° C. in a ultra-deep freezer until use.

4) For oral use, the above-described capsule was further encapsulated with a #0 capsule to give a double-layered capsule and stored in a freezer at −20° C. After leaving it at −20° C. for about an hour, it was given for oral use.

FMT using a capsule had similar effects and this means that bacteria themselves contributed to amelioration of GVHD.

### REFERENCES

1. Hirsch B E, Saraiya N, Poeth K, Schwartz R M, Epstein M E, Honig G. Effectiveness of fecal-derived microbiota transfer using orally administered capsules for recurrent Clostridium difficile infection. BMC Infect Dis. 2015; 15(1):191.
2. Youngster I, Russell G H, Pindar C, Ziv-Baran T, Sauk J, Hohmann E L. Oral, capsulized, frozen fecal microbiota transplantation for relapsing Clostridium difficile infection. JAMA. 2014; 312(17):1772-1778.

1. A composition for preventing or treating a graft-versus-host disease, the composition comprising a fecal microbiota.

2. The composition according to claim 1, wherein the fecal microbiota is contained in feces or a processed material thereof.

3. The composition according to claim 1, wherein the microorganism is a microorganism that belongs to any one genus selected from the group consisting of Lactobacillus, Bacteroides, Bifidobacterium and Faecalibacterium, Blautia, Clostridium, or a combination thereof.

4. The composition according to claim 1, wherein the graft-versus-host disease is a gastrointestinal acute graft-versus-host disease.

5. The composition according to claim 1, wherein the graft-versus-host disease is a steroid-resistant or steroid-dependent graft-versus-host disease.

6. The composition according to claim 1, which is in a form of a capsule.

7. A capsule formulation for preventing or treating a graft-versus-host disease, the formulation comprising the composition according to claim 1.

8. A method for treating a graft-versus-host disease, comprising the step of administering the composition according to claim 1 to a patient with the graft-versus-host disease.

9. A method for preventing a graft-versus-host disease, comprising the step of administering the composition according to claim 1 to a patient targeted for hematopoietic stem cell transplantation, before, after or both before and after said transplantation.

10. The method of claim 8, wherein the graft-versus-host disease is a gastrointestinal acute graft-versus-host disease.

11. The method of claim 8, wherein the graft-versus-host disease is a steroid-resistant or steroid-dependent graft-versus-host disease.
